(19) United States
(12) Patent Application Publication
(10) Pub. No.: US 2023/0047102 A1
(43) Pub. Date: Feb. 16, 2023

(54) PHARMACEUTICAL COMPOSITIONS COMPRISING N-[1-(5-CYANO-PYRIDIN-2-YLMETHYL)-1H-PYRAZOL-3-YL]-2-[4-(1-TRIFLUOROMETHYL-CYCLOPROPYL)-PHENYL]-ACETAMIDE

(71) Applicant: Idorsia Pharmaceuticals Ltd, Allachwil (CH)

(72) Inventors: Michael Ambuehl, Allschwil (CH); Nasser Benniou, Allschwil (CH); Frantz Elbaz, Allschwil (CH); Frederic Heyer, Allschwil (CH); Ibrahima Sow, Allschwil (CH)

(21) Appl. No.: 17/786,965

(22) PCT Filed: Nov. 2, 2020
(86) PCT No.: PCT/IB2020/060255

§ 371 Date: Jun. 17, 2022

(30) Foreign Application Priority Data
Dec. 20, 2019 (IB) PCT/IB2019/061192

(51) Int. Cl. A61K 31/4439; A61K 47/22; A61K 47/32; A61K 47/38; A61K 47/36; A61K 47/02; A61K 47/12; A61P 25/08

(52) U.S. Cl. CPC A61K 31/4439; A61K 47/22; A61K 47/32; A61K 47/38; A61K 47/36; A61K 47/02; A61K 47/12; A61P 25/08

(57) ABSTRACT

The present invention relates to solid pharmaceutical compositions comprising N-[1-(5-cyano-pyridin-2−ylmethyl)-1H-pyrazol-3-yl]-2-[4-(1-trifluoromethyl-cyclopropyl)-phenyl]-acetamide or pharmaceutically acceptable salt thereof. The invention further relates to methods for manufacturing said compositions and their uses for the treatment or prevention of diseases and disorders linked to T-type calcium channels such as epilepsy.

This application claims the benefit of International application PCT/IB2019/061192, filed on Dec. 20, 2019, which is incorporated herein by reference for all purpose.

The present invention relates to pharmaceutical compositions comprising, as active ingredient, the compound N-[1-(5-cyano-pyridin-2-ylmethyl)-1H-pyrazol-3-yl]-2-[4-(1-trifluoromethyl-cyclopropyl)-phenyl]acetamide, pharmaceutically acceptable salts, solvates, hydrates or morphological forms thereof (hereinafter referred to as “COMPOUND”, ACT-709478, or NBI-827104). The invention further relates to methods for manufacturing said compositions and their use as pharmaceuticals. The invention notably relates to solid pharmaceutical compositions for oral use (e.g. mini-tablets), methods for manufacturing the same and their use in treatment or prevention of diseases and disorders linked to T-type calcium channels e.g. epilepsy, notably epileptic encephalopathy with continuous spike-and-wave during sleep (CSWS).

COMPOUND is a selective and orally available triple T-type calcium channel blocker which has been described for use in the prevention/prophylaxis and/or treatment of diseases or disorders in which calcium T-type channels are involved (e.g. WO 2015/186056). A solid pharmaceutical composition for oral use comprising COMPOUND has been disclosed in WO 2018/109152. Pharmaceutical formulations comprising certain Vitamin E TPGS analogues are disclosed in WO 2006/039268. In (Yang C, et al. Theranostics. 2018 Jan 1; 8(2):464-485. doi: 10.7150/thno.22711) and (Guo Y, Eur. J. Pharm Sci. 2013 May 13; 49(2):175-86. doi: 10.1016/j.ejps.2013.02.006) the use of Vitamin E TPGS in pharmaceutical formulations is reviewed.

The clinical evaluation of COMPOUND in humans required that certain solid pharmaceutical compositions for oral delivery of COMPOUND are developed. Such compositions for oral use may allow for flexible and accurate dosing in inter a/ia pediatric patients and patients experiencing swallowing difficulties. The pharmaceutical formulations in accordance with the present invention may exhibit favorable in vitro and/or in vivo pharmacological properties such as dissolution rate profile characteristics and/or bioavailability. The compositions of the present invention may have advantageous physical properties e.g. flowability, friability and compressibility as well as other physical attributes typically being part of the quality requirements for solid oral dosage forms.

### DESCRIPTION OF THE FIGURES

FIG. 1 depicts the dissolution rate profiles (X-axis shows time in minutes; Y-axis shows percentage of dissolved COMPOUND) in gastrointestinal simulated medium of uncoated mini-tablets comprising COMPOUND (5×2 mg) and different surfactants-(Δ)-0.29% Sodium lauryl sulfate (Reference Example 5); (∘)-5% Poloxamer 188 (Reference Example 4); (□)-2.5% Vitamin E TPGS (Example 1); (▴)-4.55% Vitamin E TPGS (Example 2); and (▪)-10% Vitamin TPGS (Example 3);. The horizontal dotted line in FIG. 1 shows the intrinsic solubility of COMPOUND under the experimental conditions of the dissolution test.

FIG. 2 depicts dissolution rate profiles (X-axis shows time in minutes; Y-axis shows percentage of dissolved COMPOUND) in gastrointestinal simulated medium of (▪)-one coated mini-tablet comprising COMPOUND (1×2 mg) and 4.55% Vitamin E TPGS (Example 2); and (▴) -one coated mini-tablet comprising COMPOUND (1×2 mg) and no surfactant (Reference Example 6).

### DETAILED DESCRIPTION OF THE INVENTION

1) A first aspect of the invention relates to a solid pharmaceutical composition comprising, as active ingredient, N-[1-(5-cyano-pyridin-2-ylmethyl)-1H-pyrazol-3-yl]-2-[4-(1-trifluoromethyl-cyclopropyl)-phenyl]acetamide or a pharmaceutically acceptable salt thereof (notably the free base thereof; especially the anhydrous free base thereof), further comprising, as surfactant, tocopherol polyethylene glycol succinate.

The term “solid pharmaceutical composition” in the context of the present invention refers to solid (at room temperature) particulate matter (i.e. particles) comprising an active pharmaceutical ingredient and at least one pharmaceutical excipient different than said active pharmaceutical ingredient. Said term especially refers to a solid pharmaceutical composition for oral use. Such solid pharmaceutical composition for oral use includes, but is not limited to, a tablet, mini-tablet, micro-tablet, multiparticulate tablet, capsule-shaped tablet, chewable tablet, effervescent tablet, orodispersible tablet, pediatric tablet, cachet, capsule, chewable capsule, gastro-resistant capsule, modified-release capsule, oral lyophilizate, pellet, micro-pellet, multiple unit pellet system, bead, pill, pastille, lozenge, sphere, micro-sphere, granule, oral granule for sprinkle, effervescent granule, dragee, rod, disc, pillule, sprinkle, powder and effervescent powder. Notably, the term “solid pharmaceutical composition for oral use” in the context of the present invention refers to a tablet and especially to a mini-tablet.

The term “tablet” in the context of the present invention refer to a solid pharmaceutical composition having an oval, oblong, round, cylindrical, discoid, triangular, rectangular, hexagonal, octagonal or similar shape (notably discoid) obtained by compressing a mixture comprising at least one excipient and at least one active pharmaceutical ingredient e.g. in a suitable die via suitable punches. It is understood that the upper and lower surfaces of a tablet may be flat, round, concave or convex to various degrees.

The term “mini-tablet” refers to a tablet having a size from about 1 to about 4 mm; or equal or smaller than 2.8 mm; notably from about 2 to about 3 mm; especially having a size of up to 2.5 mm with no more than about 10% variation over this size. The term “size” as used to refer to a solid pharmaceutical composition (notably tablet or mini-tablet) means the diameter of said pharmaceutical composition, given in e.g. in millimeters.

The term “pharmaceutically acceptable salts” in the context of the present invention refers to salts which essentially retain the desired biological activity of the subject compound and exhibit minimal undesired toxicological effects. Such salts include inorganic or organic acid and/or base addition salts depending on the presence of basic and/or acidic groups in the subject compound. For reference see for example “Handbook of Pharmaceutical Salts. Properties, Selection and Use.”, P. Heinrich Stahl, Camille G. Wermuth (Eds.), Wiley-VCH, 2008; and “Pharmaceutical Salts and Co-crystals”, Johan Wouters and Luc Quere (Eds.), RSC Publishing,

The term “surfactant” in the context of the present invention refers to a surface-active agent or mixture of surface-active agents (i.e. substances active in the interface between two condensed phases i.e. solid-liquid interface) possessing both polar (hydrophilic) and non-polar (hydrophobic) parts in the same molecule.

The term “tocopherol polyethylene glycol succinate” in the context of the present invention refers to an ester of succinic acid, wherein the first carboxylic group of said acid is esterified with polyethylene glycol, said polyethylene glycol comprising a varying number of ethylene glycol units and having an average molecular weight from about 50 to about 40000; and wherein the second carboxylic group is esterified with α-tocopherol, β-tocopherol, γ-tocopherol, δ-tocopherol, or a mixture of said tocopherols (especially esterified with α-tocopherol). Notably, the term refers to said ester, wherein the first carboxylic group is esterified with polyethylene glycol having an average molecular weight of about 1000; and the second carboxylic group is esterified with an α-tocopherol. Especially, the term refers to D-α-tocopherol polyethylene glycol 1000 succinate also known as Vitamin E TPGS, which is further known in the art as poly(oxy-1,2-ethanediyl), α-[4-[[(2R)-3,4-dihydro-2, 5,7,8-tetramethyl-2-[(4 R, 8R)-4, 8, 12-trimethyltridecyl]-2 H-1-benzopyran-6-yl]oxy]-1, 4-dioxobutyl]-w-hydroxy-; poly(oxy-1,2-ethanediyl), α-[4-[[3,4-dihydro-2,5,7,8-tetramethyl-2-(4,8,12-trimethyltridecyl)-2H-1-benzopyran-6-yl]oxy]-1,4-dioxobutyl]-w-hydroxy-, [2R-[2R*(4R*,8R*)]]-; α-Tocopherol polyethylene glycol succinate; α-Tocopheryl polyethylene glycol succinate; D1T; D-α-Tocopherol acid polyethylene glycol 1000 succinate; D-α-Tocopherol polyethylene glycol 1000 succinate; d-α-Tocopheryl poly(ethylene glycol) 1000 succinate; D-α-Tocopheryl polyethylene glycol succinate; d-α-Tocopheryl polyethylene glycol succinate; Eastman Vitamin ETPGS; Ethoxylated vitamin E succinate; Kolliphor TPGS; Mono[2,5,7,8-tetramethyl-2-(4,8,12-trimethyltridecyl)-6-chromanyl]succinate, polyoxyethylene ether; Polyethylene glycol vitamin E succinate; Speziol TPGS Pharma; Tocofersolan, Tocophersolan; TPGS; TPGS 1000; TPGS 1500; TPGS 400-1000; VE-TPGS 1000; Vitamin E-TPGS and VitE-TPGS.

Vitamin E-TPGS is classified as a polar surfactant with a hydrophilic-lipophilic balance (HLB) value of about 13 subject to certain variations of this value found in the literature.

Sodium lauryl sulfate (SLS) is an anionic surfactant comprising a mixture of sodium alkyl sulfates, which according to European Pharmacopoeia 7.4 contains not less than 85% of sodium alkyl sulfates calculated as C₁₂H₂₅NaO₄S i.e. sodium dodecyl sulfate (MW=288). SLS is classified as a polar surfactant with a hydrophilic-lipophilic balance (HLB) value of about 40 subject to certain variations of this value found in the literature. SLS is used a commonly used excipient in solid pharmaceutical formulations such as tablets typically in the range 0.2 to 1.5 ww % (see e.g. European Medicines Agency EMA/CHMP/351898/2014).

Poloxamer 188 (such as Kolliphor® P188) is a non-ionic block linear copolymer composed of two hydrophilic side-chains attached to a hydrophobic central core. Poloxamer 188 is classified as a polar surfactant with a hydrophilic-lipophilic balance (HLB) value about 29 subject to certain variations of this value found in the literature.

Surfactants, especially commercially available surfactant products such as Vitamin E TPGS, may not be pure compounds but rather mixtures of compounds containing one primary major surfactant component such as the polyethylene glycol succinate of tocopherol (notably the D-α-tocopherol polyethylene glycol 1000 succinate) and may contain variable amounts of said primary major surfactant component and residual amounts of further components such as free ethylene glycol, free polyethylene glycol chains of varying length, succinic acid mono- or diesters of α-tocopherol, or succinic acid mono- or diesters of polyethylene glycol; as well as variable amounts of solvents, such as water or organic solvents and traces of metals. The above-mentioned residual further components are encompassed in the scope of term “polyethylene glycol succinate of tocopherol” (notably in the scope of the term “D-α-tocopherol polyethylene glycol 1000 succinate” also known as Vitamin E TPGS) as used herein.

2) Another embodiment relates to the composition according to embodiment 1), wherein the active ingredient is from about 5 to about 60 ww % (notably from about 10 to about 40 ww %; especially from about 20 to about 30 ww %; in particular about 26 ww %).

3) Another embodiment relates to the composition according to embodiment 1) or 2), wherein the surfactant is α-tocopherol polyethylene glycol succinate (notably D-α-tocopherol polyethylene glycol 1000 succinate (i.e. Vitamin E TPGS)).

4) Another embodiment relates to the composition according to any one of embodiments 1) to 3), wherein the surfactant is from about 0.1 to about 20 ww % (notably from about 1 to about 10 ww %; especially from about 2 to about 6 ww %; in particular about 4.5 ww %) [In a sub-embodiment of embodiment 4) said composition further comprises at least one solid pharmaceutical excipient].

5) Another embodiment relates to the composition according to any one of embodiments 1) to 4), further comprising one or more fillers (in particular, microcrystalline cellulose) (notably from about 20 to about 60 ww %; especially from about 30 to about 40 ww %; in particular about 34 ww %).

6) Another embodiment relates to the composition according to any one of embodiments 1) to 5), further comprising one or more disintegrants (notably from about 2 to about 40 ww %; especially from about 10 to about 20 ww %; in particular about 17 ww %).

7) Another embodiment relates to the composition according to any one of embodiments 1) to 6), further comprising one or more binders (notably from about 0 to about 10 ww % of the total composition; especially from about 3 to about 7 ww % of the total composition; in particular about 4.6 ww %).

8) Another embodiment relates to the composition according to any one of embodiments 1) to 7), further comprising one or more glidants (notably from about 0 to about 1 ww %; especially from about 0.1 to about 0.5 ww %; in particular about 0.2 ww %).

9) Another embodiment relates to the composition according to any one of embodiments 1) to 8), further comprising one or more lubricants (notably from about 0 to about 5 ww %; especially from about 0.1 to about 0.5 ww %; in particular about 0.2 ww %).

10) Another embodiment relates to the composition according to any one of embodiments 1) to 9), further comprising one or more coating agents (notably from about 0 to about 20 ww %; especially from about 5 to about 15 ww %; in particular about 9 ww %).

11) Another embodiment relates to a solid pharmaceutical composition according to embodiment 1) comprising
N-[1-(5-cyano-pyridin-2-ylmethyl)-1H-pyrazol-3-yl]-2-[4-(1-trifluoromethyl-cyclopropyl)-phenyl]-acetamide or a pharmaceutically acceptable salt thereof (notably the free base thereof; especially a crystalline form of the free base thereof) (notably from about 10 to about 40 ww % of the total composition);
tocopherol polyethylene glycol succinate (notably from about 1 to about 10 ww % of the total composition); and
one or more further excipients such as fillers, binders, disintegrants, glidants, lubricants and/or coating agents (notably wherein said fillers, binders, disintegrants, glidants, lubricants and/or coating agents are in amounts according to the corresponding embodiments 5) to 10);

wherein the total of said pharmaceutical composition is 100 ww %.

12) Another embodiment relates to a solid pharmaceutical composition according to embodiment 1) comprising
from about 20 to about 30 ww % N-[1-(5-cyano-pyridin-2-ylmethyl)-1H-pyrazol-3-yl]-2-[4-(1-trifluoromethyl-cyclopropyl)-phenyl]-acetamide or a pharmaceutically acceptable salt thereof (notably the free base thereof; especially a crystalline form of the free base thereof);
from about 0.5 to about 10 ww % D-α-tocopherol polyethylene glycol 1000 succinate;
from about 20 to about 40 ww % microcrystalline cellulose;
from about 1 to about 10 ww % polyvinylpyrrolidone;
from about 5 to about 25 ww % partially pregelatinized maize starch;
from 0 to about 3 ww % hydrophilic fumed silica;
from 0 to about 3 ww % magnesium stearate; and
from 0 to about 15 ww % coating agent;

wherein the total of said pharmaceutical composition is 100 ww %.

13) Another embodiment relates to a solid pharmaceutical composition according to embodiment 1) comprising
from about 24 to about 28 ww % N-[1-(5-cyano-pyridin-2-ylmethyl)-1H-pyrazol-3-yl]-2-[4-(1-trifluoromethyl-cyclopropyl)-phenyl]acetamide or a pharmaceutically acceptable salt thereof (notably the free base thereof; especially a crystalline form of the free base thereof);
from about 3.6 to about 5.6 ww % D-α-tocopherol polyethylene glycol 1000 succinate;
from about 33 to about 38 ww % microcrystalline cellulose;
from about 3 to about 7 ww % polyvinylpyrrolidone;
from about 10 to about 20 ww % partially pregelatinized maize starch;
from about 0.1 to about 0.5 ww % hydrophilic fumed silica;
from about 0.1 to about 0.5 ww % magnesium stearate; and
from about 7 to about 12 ww % coating agent;

wherein the total of said pharmaceutical composition is 100 ww %.

14) Another embodiment relates to a solid pharmaceutical composition according to embodiment 1) consisting essentially of
from about 20 to about 30 ww % N-[1-(5-cyano-pyridin-2-ylmethyl)-1H-pyrazol-3-yl]-2-[4-(1-trifluoromethyl-cyclopropyl)-phenyTacetamide or a pharmaceutically acceptable salt thereof (notably the free base thereof; especially a crystalline form of the free base thereof);
from about 2 to about 6 ww % D-α-tocopherol polyethylene glycol 1000 succinate; and
from about 64 to about 78 ww % one or more further excipients (notably one or more fillers (especially microcrystalline cellulose), one or more binders (especially polyvinylpyrrolidone), disintegrants (especially partially pregelatinized maize starch), lubricants (especially hydrophilic fumed silica), glidants (especially magnesium stearate) and/or coating agents)

wherein the total of said pharmaceutical composition is 100 ww %.

15) Another embodiment relates to a solid pharmaceutical composition according to embodiment 1) consisting essentially of
about 26 ww % N-[1-(5-cyano-pyridin-2-ylmethyl)-1H-pyrazol-3-yl]-2-[4-(1-trifluoromethyl-cyclopropyly phenyTacetamide or a pharmaceutically acceptable salt thereof (notably the free base thereof; especially a crystalline form of the free base thereof);
about 4.5 ww % D-α-tocopherol polyethylene glycol 1000 succinate;
about 34 ww % microcrystalline cellulose;
about 9 ww % polyvinylpyrrolidone;
about 17 ww % partially pregelatinized maize starch;
about 0.25 ww % of one or more lubricants (notably hydrophilic fumed silica);
about 0.25 ww % of one or more glidants (notably magnesium stearate); and
about 9 ww % of one or more coating agents.

16) Another aspect of the invention relates to the composition according to any one of embodiments 1) to 15), wherein the pharmaceutical composition is in the form of a tablet, mini-tablet, micro-tablet, capsule-shaped tablet, caplet, pediatric tablet, cachet, capsule, pellet, micro-pellet, bead, pill, sphere, micro-sphere, granule, oral granule, dragee, rod, disc (notably tablet, mini-tablet, micro-tablet, pediatric tablet, pellet, micro-pellet, bead, pill, sphere, micro-sphere or granule; especially tablet or mini-tablet; in particular mini-tablet).

17) Another embodiment relates to the composition according to embodiment 16), wherein the pharmaceutical composition has a size from about 1 to about 4 mm (notably a size from about 2 to about 3 mm; especially a size equal or smaller than 2.8 mm; in particular a size of up to 2.5 mm with no more than about 10% variation over this size).

The term “mini-tablets” commonly refers to compressed tablets with size smaller than typical tablets. Although there are currently no regulatory guidelines that define mini-tablets, the term has been used to describe tablets with diameters between one to four millimeters. Since oral dosage forms smaller than 2.5 mm can be considered as oral granules, many mini-tablet products are focused at this size range, to take advantage of the potential flexibility in dosage form administration (e.g. mixed with soft foods). For more details, see https://www.americanpharmaceuticalreview.com/Featured-Articles/190921-Minitablets-Manufacturing-Charac terization-Methods-and-Future-Opportunities/.

18) Another embodiment relates to the composition according to embodiment 16) or 17), wherein the composition has a size suitable for administration in pediatric patients (notably patients of or younger than 18 years of age; especially patients of or younger than 12 years of age; in particular patients of or younger than 6 years of age;) and/or in patients experiencing (or diagnosed with) swallowing difficulties.

19) Another embodiment relates to the composition according to embodiments 16) to 18), wherein the pharmaceutical composition has a total weight from about 1 to about 30 milligrams (notably from about 2 to about 20 milligrams; especially from about 3 to about 10 milligrams; in particular about 7 milligrams).

Based on the dependencies of the different embodiments 1) to 19) as disclosed hereinabove, the following embodiments are thus possible and intended and herewith specifically disclosed in individualized form:

2+1, 3+1, 3+2+1, 4+1, 4+2+1, 4+3+1, 4+3+2+1, 5+1, 5+2+1, 5+3+1, 5+3+2+1, 5+4+1, 5+4+2+1, 5+4+3+1, 5+4+3+2+1, 6+1, 6+2+1, 6+3+1, 6+3+2+1, 6+4+1, 6+4+2+1, 6+4+3+1, 6+4+3+2+1, 6+5+1, 6+5+2+1, 6+5+3+1, 6+5+3+2+1, 6+5+4+1, 6+5+4+2+1, 6+5+4+3+1, 6+5+4+3+2+1, 7+1, 7+2+1, 7+3+1, 7+3+2+1, 7+4+1, 7+4+2+1, 7+4+3+1, 7+4+3+2+1, 7+5+1, 7+5+2+1, 7+5+3+1, 7+5+3+2+1, 7+5+4+1, 7+5+4+2+1, 7+5+4+3+1, 7+5+4+3+2+1, 7+6+1, 7+6+2+1, 7+6+3+1, 7+6+3+2+1, 7+6+4+1, 7+6+4+2+1, 7+6+4+3+1, 7+6+4+3+2+1, 7+6+5+1, 7+6+5+2+1, 7+6+5+3+1, 7+6+5+3+2+1, 7+6+5+4+1, 7+6+5+4+2+1, 7+6+5+4+3+1, 7+6+5+4+3+2+1, 8+1, 8+2+1, 8+3+1, 8+3+2+1, 8+4+1, 8+4+2+1, 8+4+3+1, 8+4+3+2+1, 8+5+1, 8+5+2+1, 8+5+3+1, 8+5+3+2+1, 8+5+4+1, 8+5+4+2+1, 8+5+4+3+1, 8+5+4+3+2+1, 8+6+1, 8+6+2+1, 8+6+3+1, 8+6+3+2+1, 8+6+4+1, 8+6+4+2+1, 8+6+4+3+1, 8+6+4+3+2+1, 8+6+5+1, 8+6+5+2+1, 8+6+5+3+1, 8+6+5+3+2+1, 8+6+5+4+1, 8+6+5+4+2+1, 8+6+5+4+3+1, 8+6+5+4+3+2+1, 8+7+1, 8+7+2+1, 8+7+3+1, 8+7+3+2+1, 8+7+4+1, 8+7+4+2+1, 8+7+4+3+1, 8+7+4+3+2+1, 8+7+5+1, 8+7+5+2+1, 8+7+5+3+1, 8+7+5+3+2+1, 8+7+5+4+1, 8+7+5+4+2+1, 8+7+5+4+3+1, 8+7+5+4+3+2+1, 8+7+6+1, 8+7+6+2+1, 8+7+6+3+1, 8+7+6+3+2+1, 8+7+6+4+1, 8+7+6+4+2+1, 8+7+6+4+3+1, 8+7+6+4+3+2+1, 8+7+6+5+1, 8+7+6+5+2+1, 8+7+6+5+3+1, 8+7+6+5+3+2+1, 8+7+6+5+4+1, 8+7+6+5+4+2+1, 8+7+6+5+4+3+1, 8+7+6+5+4+3+2+1, 9+1, 9+2+1, 9+3+1, 9+3+2+1, 9+4+1, 9+4+2+1, 9+4+3+1, 9+4+3+2+1, 9+5+1, 9+5+2+1, 9+5+3+1, 9+5+3+2+1, 9+5+4+1, 9+5+4+2+1, 9+5+4+3+1, 9+5+4+3+2+1, 9+6+1, 9+6+2+1, 9+6+3+1, 9+6+3+2+1, 9+6+4+1, 9+6+4+2+1, 9+6+4+3+1, 9+6+4+3+2+1, 9+6+5+1, 9+6+5+2+1, 9+6+5+3+1, 9+6+5+3+2+1, 9+6+5+4+1, 9+6+5+4+2+1, 9+6+5+4+3+1, 9+6+5+4+3+2+1, 9+7+1, 9+7+2+1, 9+7+3+1, 9+7+3+2+1, 9+7+4+1, 9+7+4+2+1, 9+7+4+3+1, 9+7+4+3+2+1, 9+7+5+1, 9+7+5+2+1, 9+7+5+3+1, 9+7+5+3+2+1, 9+7+5+4+1, 9+7+5+4+2+1, 9+7+5+4+3+1, 9+7+5+4+3+2+1, 9+7+6+1, 9+7+6+2+1, 9+7+6+3+1, 9+7+6+3+2+1, 9+7+6+4+1, 9+7+6+4+2+1, 9+7+6+4+3+1, 9+7+6+4+3+2+1, 9+7+6+5+1, 9+7+6+5+2+1, 9+7+6+5+3+1, 9+7+6+5+3+2+1, 9+7+6+5+4+1, 9+7+6+5+4+2+1, 9+7+6+5+4+3+1, 9+7+6+5+4+3+2+1, 9+8+1, 9+8+2+1, 9+8+3+1, 9+8+3+2+1, 9+8+4+1, 9+8+4+2+1, 9+8+4+3+1, 9+8+4+3+2+1, 9+8+5+1, 9+8+5+2+1, 9+8+5+3+1, 9+8+5+3+2+1, 9+8+5+4+1, 9+8+5+4+2+1, 9+8+5+4+3+1, 9+8+5+4+3+2+1, 9+8+6+1, 9+8+6+2+1, 9+8+6+3+1, 9+8+6+3+2+1, 9+8+6+4+1, 9+8+6+4+2+1, 9+8+6+4+3+1, 9+8+6+4+3+2+1, 9+8+6+5+1, 9+8+6+5+2+1, 9+8+6+5+3+1, 9+8+6+5+3+2+1, 9+8+6+5+4+1, 9+8+6+5+4+2+1, 9+8+6+5+4+3+1, 9+8+6+5+4+3+2+1, 9+8+7+1, 9+8+7+2+1, 9+8+7+3+1, 9+8+7+3+2+1, 9+8+7+4+1, 9+8+7+4+2+1, 9+8+7+4+3+1, 9+8+7+4+3+2+1, 9+8+7+5+1, 9+8+7+5+2+1, 9+8+7+5+3+1, 9+8+7+5+3+2+1, 9+8+7+5+4+1, 9+8+7+5+4+2+1, 9+8+7+5+4+3+1, 9+8+7+5+4+3+2+1, 9+8+7+6+1, 9+8+7+6+2+1, 9+8+7+6+3+1, 9+8+7+6+3+2+1, 9+8+7+6+4+1, 9+8+7+6+4+2+1, 9+8+7+6+4+3+1, 9+8+7+6+4+3+2+1, 9+8+7+6+5+1, 9+8+7+6+5+2+1, 9+8+7+6+5+3+1, 9+8+7+6+5+3+2+1, 9+8+7+6+5+4+1, 9+8+7+6+5+4+2+1, 9+8+7+6+5+4+3+1, 9+8+7+6+5+4+3+2+1, 10+1, 10+2+1, 10+3+1, 10+3+2+1, 10+4+1, 10+4+2+1, 10+4+3+1, 10+4+3+2+1, 10+5+1, 10+5+2+1, 10+5+3+1, 10+5+3+2+1, 10+5+4+1, 10+5+4+2+1, 10+5+4+3+1, 10+5+4+3+2+1, 10+6+1, 10+6+2+1, 10+6+3+1, 10+6+3+2+1, 10+6+4+1, 10+6+4+2+1, 10+6+4+3+1, 10+6+4+3+2+1, 10+6+5+1, 10+6+5+2+1, 10+6+5+3+1, 10+6+5+3+2+1, 10+6+5+4+1, 10+6+5+4+2+1, 10+6+5+4+3+1, 10+6+5+4+3+2+1, 10+7+1, 10+7+2+1, 10+7+3+1, 10+7+3+2+1, 10+7+4+1, 10+7+4+2+1, 10+7+4+3+1, 10+7+4+3+2+1, 10+7+5+1, 10+7+5+2+1, 10+7+5+3+1, 10+7+5+3+2+1, 10+7+5+4+1, 10+7+5+4+2+1, 10+7+5+4+3+1, 10+7+5+4+3+2+1, 10+7+6+1, 10+7+6+2+1, 10+7+6+3+1, 10+7+6+3+2+1, 10+7+6+4+1, 10+7+6+4+2+1, 10+7+6+4+3+1, 10+7+6+4+3+2+1, 10+7+6+5+1, 10+7+6+5+2+1, 10+7+6+5+3+1, 10+7+6+5+3+2+1, 10+7+6+5+4+1, 10+7+6+5+4+2+1, 10+7+6+5+4+3+1, 10+7+6+5+4+3+2+1, 10+8+1, 10+8+2+1, 10+8+3+1, 10+8+3+2+1, 10+8+4+1, 10+8+4+2+1, 10+8+4+3+1, 10+8+4+3+2+1, 10+8+5+1, 10+8+5+2+1, 10+8+5+3+1, 10+8+5+3+2+1, 10+8+5+4+1, 10+8+5+4+2+1, 10+8+5+4+3+1, 10+8+5+4+3+2+1, 10+8+6+1, 10+8+6+2+1, 10+8+6+3+1, 10+8+6+3+2+1, 10+8+6+4+1, 10+8+6+4+2+1, 10+8+6+4+3+1, 10+8+6+4+3+2+1, 10+8+6+5+1, 10+8+6+5+2+1, 10+8+6+5+3+1, 10+8+6+5+3+2+1, 10+8+6+5+4+1, 10+8+6+5+4+2+1, 10+8+6+5+4+3+1, 10+8+6+5+4+3+2+1, 10+8+7+1, 10+8+7+2+1, 10+8+7+3+1, 10+8+7+3+2+1, 10+8+7+4+1, 10+8+7+4+2+1, 10+8+7+4+3+1, 10+8+7+4+3+2+1, 10+8+7+5+1, 10+8+7+5+2+1, 10+8+7+5+3+1, 10+8+7+5+3+2+1, 10+8+7+5+4+1, 10+8+7+5+4+2+1, 10+8+7+5+4+3+1, 10+8+7+5+4+3+2+1, 10+8+7+6+1, 10+8+7+6+2+1, 10+8+7+6+3+1, 10+8+7+6+3+2+1, 10+8+7+6+4+1, 10+8+7+6+4+2+1, 10+8+7+6+4+3+1, 10+8+7+6+4+3+2+1, 10+8+7+6+5+1, 10+8+7+6+5+2+1, 10+8+7+6+5+3+1, 10+8+7+6+5+3+2+1, 10+8+7+6+5+4+1, 10+8+7+6+5+4+2+1, 10+8+7+6+5+4+3+1, 10+8+7+6+5+4+3+2+1, 10+9+1, 10+9+2+1, 10+9+3+1, 10+9+3+2+1, 10+9+4+1, 10+9+4+2+1, 10+9+4+3+1, 10+9+4+3+2+1, 10+9+5+1, 10+9+5+2+1, 10+9+5+3+1, 10+9+5+3+2+1, 10+9+5+4+1, 10+9+5+4+2+1, 10+9+5+4+3+1, 10+9+5+4+3+2+1, 10+9+6+1, 10+9+6+2+1, 10+9+6+3+1, 10+9+6+3+2+1, 10+9+6+4+1, 10+9+6+4+2+1, 10+9+6+4+3+1, 10+9+6+4+3+2+1, 10+9+6+5+1, 10+9+6+5+2+1, 10+9+6+5+3+1, 10+9+6+5+3+2+1, 10+9+6+5+4+1, 10+9+6+5+4+2+1, 10+9+6+5+4+3+1, 10+9+6+5+4+3+2+1, 10+9+7+1, 10+9+7+2+1, 10+9+7+3+1, 10+9+7+3+2+1, 10+9+7+4+1, 10+9+7+4+2+1, 10+9+7+4+3+1, 10+9+7+4+3+2+1, 10+9+7+5+1, 10+9+7+5+2+1, 10+9+7+5+3+1, 10+9+7+5+3+2+1, 10+9+7+5+4+1, 10+9+7+5+4+2+1, 10+9+7+5+4+3+1, 10+9+7+5+4+3+2+1, 10+9+7+6+1, 10+9+7+6+2+1, 10+9+7+6+3+1, 10+9+7+6+3+2+1, 10+9+7+6+4+1, 10+9+7+6+4+2+1, 10+9+7+6+4+3+1, 10+9+7+6+4+3+2+1, 10+9+7+6+5+1, 10+9+7+6+5+2+1, 10+9+7+6+5+3+1, 10+9+7+6+5+3+2+1, 10+9+7+6+5+4+1, 10+9+7+6+5+4+2+1, 10+9+7+6+5+4+3+1, 10+9+7+6+5+4+3+2+1, 10+9+8+1, 10+9+8+2+1, 10+9+8+3+1, 10+9+8+3+2+1, 10+9+8+4+1, 10+9+8+4+2+1, 10+9+8+4+3+1, 10+9+8+4+3+2+1, 10+9+8+5+1, 10+9+8+5+2+1, 10+9+8+5+3+1, 10+9+8+5+3+2+1, 10+9+8+5+4+1, 10+9+8+5+4+2+1, 10+9+8+5+4+3+1, 10+9+8+5+4+3+2+1, 10+9+8+6+1, 10+9+8+6+2+1, 10+9+8+6+3+1, 10+9+8+6+3+2+1, 10+9+8+6+4+1, 10+9+8+6+4+2+1, 10+9+8+6+4+3+1, 10+9+8+6+4+3+2+1, 10+9+8+6+5+1, 10+9+8+6+5+2+1, 10+9+8+6+5+3+1, 10+9+8+6+5+3+2+1, 10+9+8+6+5+4+1, 10+9+8+6+5+4+2+1, 10+9+8+6+5+4+3+1, 10+9+8+6+5+4+3+2+1, 10+9+8+7+1, 10+9+8+7+2+1, 10+9+8+7+3+1, 10+9+8+7+3+2+1, 10+9+8+7+4+1, 10+9+8+7+4+2+1, 10+9+8+7+4+3+1, 10+9+8+7+4+3+2+1, 10+9+8+7+5+1, 10+9+8+7+5+2+1, 10+9+8+7+5+3+1, 10+9+8+7+5+3+2+1, 10+9+8+7+5+4+1, 10+9+8+7+5+4+2+1, 10+9+8+7+5+4+3+1, 10+9+8+7+5+4+3+2+1, 10+9+8+7+6+1, 10+9+8+7+6+2+1, 10+9+8+7+6+3+1, 10+9+8+7+6+3+2+1, 10+9+8+7+6+4+1, 10+9+8+7+6+4+2+1, 10+9+8+7+6+4+3+1, 10+9+8+7+6+4+3+2+1, 10+9+8+7+6+5+1, 10+9+8+7+6+5+2+1, 10+9+8+7+6+5+3+1, 10+9+8+7+6+5+3+2+1, 10+9+8+7+6+5+4+1, 10+9+8+7+6+5+4+2+1, 10+9+8+7+6+5+4+3+1, or 10+9+8+7+6+5+4+3+2+1.

The term “filler” also referred as “bulking agent” or “diluent” refers in the context of the present invention to a pharmaceutical excipient added to a solid pharmaceutical formulation comprising a lower amount of active ingredient in order to increase their size/weight, thereby allowing for better handling and compression. Suitable fillers within the scope of the present invention are lactose, microcrystalline cellulose, mannitol, maltitol, maltodextrin, dicalcium phosphate, dibasic calcium phosphate dihydrate (CaHPO₄·2H₂O), calcium sulfate, starch, cellulose, kaolin, sodium chloride, anhydrous lactose, sorbitol, sucrose, or a mixture thereof; notably microcrystalline cellulose (such as Avicel™ PH-101 or PH-102).

The term “binder” in the context of the present invention refers to a pharmaceutical excipient which holds the ingredients in a tablet and/or granule together ensuring that tablet and/or granule can be formed with the required mechanical strength. Suitable binders within the scope of the present invention are corn starch, maize starch, gelatin, acacia gum, guar gum, xanthan gum, hydroxypropylmethylcellulose, tragacanth gum, polyvinylpyrrolidone (PVP), hydroxy methyl cellulose, or a mixture thereof; notably polyvinylpyrrolidone also known as povidone such as PVP K30.

The term “disintegrant” in the context of the present invention refers to a pharmaceutical excipient which expands when wet causing a tablet or granule to break down into smaller fragments in the digestive tract (or in specific segments thereof), releasing the active ingredients for absorption. Suitable disintegrants within the scope of the present invention are corn starch, potato starch, sodium starch glycolate, pregelatinized maize starch, alginic acid, sodium alginate, agar, bentonite, sodium carboxymethylcellulose, calcium carboxymethylcellulose, croscarmellose sodium, cross-linked polyvinylpyrrolidone (e.g. Crospovidone (PVP XL; Polyplasdone, commercially available from the ISP company or Kollidon® XL from BASF)), clays, or a mixture thereof; notably partially pregelatinized maize starch such as Starch 1500™

The term “glidant” in the context of the present invention refers to a pharmaceutical excipient which facilitates powder flow by reducing interparticle friction and cohesion. Suitable glidants within the scope of the present invention are silicon dioxide, hydrophilic fumed silica, talc, magnesium trisilicate, powdered cellulose, magnesium carbonate, or a mixture thereof; notably hydrophilic fumed silica such as Aerosil™200.

The term “lubricant” in the context of the present invention refers to a pharmaceutical excipient which prevents pharmaceutical ingredients from clumping together and from sticking to parts of manufacturing equipment (e.g. tableting or filling equipment) such as punches, dies, etc. Suitable lubricants within the scope of the present invention are polyoxyethylene stearic acid, stearic acid, stearic acid salts such as Mg-, Al- or Ca-stearate, lauryl sulphate salts, sodium stearyl fumarate, glyceryl behenate, glyceryl mono fatty acid e.g. having a molecular weight of from 200 to 800 Daltons (e.g. glyceryl monostearate (e.g. from Danisco, UK)), glyceryl dibehenate (e.g. Compritol AT0888™, Gattefossé France), glyceryl palmito-stearic ester (e.g. Precirol™, Gattefossé France), polyethylene glycol (PEG, BASF), hydrogenated cotton seed oil (Lubitab, Edward Mendell Co Inc.), hydrogenated castor seed oil (Cutina HR, Henkel) and; glyceryl palmitostearate, hydrogenated vegetable oils, talc, sodium benzoate, or a mixture thereof; notably magnesium stearate such as Parteck™ LUB MST.

The term “coating agent” in the context of the present invention refers to a pharmaceutical excipient which is used to film coat the solid pharmaceutical compositions in order to protect the active ingredient from e.g. moisture, light, the acidic environment of the stomach, or especially to mask the taste of bad tasting actives.

Coating agents suitable for use in the compositions of the present invention are made of cellulose derivatives such as methyl or ethyl cellulose, sodium carboxy methyl cellulose, hydroxypropyl cellulose, hydroxy propyl methyl cellulose, hydroxy propyl methyl cellulose phthalate, methacrylic acid copolymers, polyethylene glycol, triacetin, iron oxides, colorants, talc, titanium dioxide, or a mixture thereof; notably one or more film-forming cellulose ethers with plasticizing and coloring additives (such as AquaPolish™ P white MS).

Where the plural form is used in terms such as “compounds”, “salts”, “compositions”, “formulations”, “dosage forms”, “excipients”, “diseases”, “conditions” and the like, it is intended to mean also the single form “compound”, “salt”, “composition”, “formulation”, “dosage form”, “excipient”, “disease”, “condition” and the like.

Any reference to COMPOUND is to be understood as referring also to the pharmaceutically acceptable salts of such compound. Notably, COMPOUND refers to the free base; especially to anhydrous free base.

COMPOUND of the present invention may be a crystalline material of one or more polymorphic modifications. Also, it may be an amorphous material, or a mixture of crystalline material of one or more polymorphic modifications and amorphous material. It is further understood that crystalline forms of COMPOUND encompass all types of crystalline forms of COMPOUND including crystalline forms of the mere molecule, of solvates or hydrates, of molecular salts or of co-crystals (when the same molecule can be co-crystallized with different co-crystal formers) provided they are suitable for pharmaceutical administration.

COMPOUND of the present invention is a notably a crystalline material; especially COMPOUND is in crystalline from 1 or 2 (especially 1) as described in WO 2019/008034.

It is understood, that the crystalline form used in the compositions of the present invention comprises COMPOUND in a crystalline form which can be a crystalline form of the COMPOUND in free base form; a crystalline form of the COMPOUND in free base form wherein said crystalline form is a cocrystal, or a crystalline form of the COMPOUND in form of a pharmaceutically acceptable salt, or a solvate of any of such forms. Furthermore, said crystalline forms may comprise non-coordinated and/or coordinated solvent. Coordinated solvent is used herein as term for a crystalline solvate. Likewise, non-coordinated solvent is used herein as term for physiosorbed or physically entrapped solvent (definitions according to Polymorphism in the Pharmaceutical Industry (Ed. R. Hilfiker, V C H, 2006), Chapter 8: U. J. Griesser: The Importance of Solvates). Such crystalline form may be especially an anhydrite, i.e. it comprises no significant amounts of coordinated water.

The present compositions encompass COMPOUND in essentially pure form. The ww % amount of COMPOUND may need to be adjusted in view of the actual chemical purity of the material, the presence of: a co-crystal former, a salt former (such as an acid), a solvate, or a hydrate.

The pharmaceutical compositions according to the present invention may be filled in containers. Any type of containers suitable to contain pharmaceutical compositions in solid form (such as tablets or mini-tablets) may be used in the present invention. Examples of such containers are bottles, vials, or tubes. Notably, plastic containers such as containers made of high-density polyethylene or glass may be used.

The pharmaceutical compositions according to the present invention may further be provided to patients using standard packaging or devices known in the art such as blister packs, stripe-packs, stick-packs, sachets, pouches, bags, capsules, manually openable capsules, bottles, containers, dispensers, droppers or boxes as well as known in the art mechanical and/or electronic devices suitable for controlled dosing of the compositions of the present invention. Such packaging or devices may be useful to control and deliver the required dosage, especially in pediatric populations or populations having swallowing difficulties.

For avoidance of any doubt, it is well understood that the pharmaceutical composition as defined in any one of embodiments 1 to 19) may additionally comprise further conventional excipients, ingredients and/or additives, which may be used alone or in combination (quantum satis, i.e. wherein the maximum amounts of said further conventional ingredients or additives and/or the maximum amounts of the respective mixture of excipients may need to be reduced to make up the total ww % of 100).

The compositions of the present invention may comprise further pharmaceutical excipients. Reference is made to the extensive literature on the subject, see for example R. C. Rowe, P. J. Seskey, S. C. Owen, Handbook of Pharmaceutical Excipients, 5th edition, Pharmaceutical Press 2006; Remington, The Science and Practice of Pharmacy, 21st Edition (2005), Part 5, “Pharmaceutical Manufacturing” [published by Lippincott Williams & Wilkins]. It is understood that the excipients used herein comply with at least one of the following Japanese Pharmacopoeia; European Pharmacopoeia; United States Pharmacopoeia; United States Pharmacopoeia/National Formulary.

Unless used regarding temperatures, the term “about” placed before a numerical value “X” refers in the current application to an interval extending from X minus 10% of X to X plus 10% of X, and preferably to an interval extending from X minus 5% of X to X plus 5% of X (wherein it is well understood that values below 0%, respectively higher than 100%, are not applicable). In case the term about is placed before a range, the respective interval is to be applied to both values of the range. In the particular case of temperatures, the term “about” placed before a temperature “Y” refers in the current application to an interval extending from the temperature Y minus 10° C. to Y plus 10° C.; and preferably, in case the temperature is at least 30° C. to an interval extending from Y minus 5° C. to Y plus 5° C.; or, in case the temperature is below 30° C., to an interval extending from Y minus 2° C. to Y plus 2° C. Room temperature means a temperature of about 25° C.

The term “consisting essentially of” is understood in the context of the present invention to mean especially that the respective composition consists in an amount of at least 90, notably of at least 95, especially of at least 99, and preferably in an amount of 100 percent by weight (i.e. in the meaning of “consisting of”) of the respective composition in the amounts as explicitly stated in the respective embodiment.

The term “essentially”, for example when used in a term such as “essentially pure” is understood in the context of the present invention to mean especially that the respective composition/compound etc. consists in an amount of at least 90, notably of at least 95, and especially of at least 99 percent by weight of the respective pure composition/compound.

The expression ww % refers to a percentage by weight of the total weight of the composition considered. If not explicitly stated otherwise, the considered total weight is the total weight of the pharmaceutical composition which is the composition including the active ingredient. It is understood that the total amount expressed in “ww %” of a certain pharmaceutical composition is 100.

For avoidance of any doubt, ww % quantities refer to the total of the respective (commercially available) excipient or active substance as added to the pharmaceutical composition; and are calculated with respect to the total weight of the pharmaceutical composition. Thus, it is understood that for calculating ww % amounts of a certain excipient, any residual substance such as polyethylene glycol, solvent(s) or other chemicals/traces, which may be present in such excipient are considered as being part of said excipient and take part in the ww % of such excipient. In case a certain value is given as % value, in absence of further specification such value refers to ww %.

It is further understood that the pharmaceutical composition as defined herein may, if not explicitly stated otherwise, additionally comprise conventional ingredients or additives (quantum satis, i.e. wherein the amounts of the mixture of excipients may need to be adjusted to the amount of said conventional ingredients or additives present in the pharmaceutical composition to make up the total ww % of 100 of the pharmaceutical composition). Preferably the total amount of such additional conventional ingredients or additives is 0 ww % to a total maximum of about 5 ww % (especially 0 ww % to a total of about 2 ww %).

The term “pharmaceutical composition(s)” is interchangeable with the terms “pharmaceutical formulation(s)” or “pharmaceutical preparation(s)”.

The pharmaceutical compositions according to the invention may be used as medicaments (notably in pediatric patients (children) and/or in patients experiencing (or diagnosed by a medical practitioner with) swallowing difficulties). Pediatric patients as defined herein are patients (humans) of or younger than 18 years of age; especially patients of or younger than 12 years of age; in particular patients of or younger than 5 years of age;

The pharmaceutical compositions as defined in any one of embodiments 1) to 19) are useful for the prevention or treatment of diseases or disorders where calcium T channels are involved. Such diseases or disorders may be defined as including especially: epilepsy (notably absence epilepsy, epileptic encephalopathy with continuous spike-and-wave during sleep (CSWS), childhood absence and other forms of idiopathic generalized epilepsies, temporal lobe epilepsy); sleep disorders and sleep disturbances; pain (notably inflammatory pain, neuropathic pain, peripheral pain, chronic pain associated with peripheral axonal injury); neurological diseases and disorders (notably essential tremor, Parkinson's disease, schizophrenia, depression, anxiety, psychosis, neurodegenerative disorders, autism, drug addiction); cardiovascular diseases and disorders (notably hypertension, cardiac arrhythmias, atrial fibrillation, congenital heart failure, heart block); cancer; diabetes and diabetic neuropathy; and infertility and sexual dysfunction. Notably, the pharmaceutical compositions of the current invention are useful for the prevention or treatment of epilepsy, neurological disorders and pain; especially epilepsy (e.g. epileptic encephalopathy with continuous spike-and-wave during sleep, also referred to as CSWS) and essential tremor.

The term “epilepsy” describes recurrent unprovoked seizures wherein the term “seizure” refers to an excessive and/or hypersynchronous electrical neuronal activity. Different types of “epilepsy” are disclosed for example in [Berg et al., Epilepsia. 2010; 51(4): 676-685], which reference is herewith incorporated by reference. The term “epilepsy” as used herein preferably refers to absence epilepsy, childhood absence and other forms of idiopathic generalized epilepsies, temporal lobe epilepsy.

The term “pain” preferably refers to inflammatory pain, neuropathic pain, peripheral pain, and chronic pain associated with peripheral axonal injury.

The term “neurological diseases and disorders” preferably refers to essential tremors, Parkinson's disease, schizophrenia, depression, anxiety, psychosis, neurodegenerative disorders, autism, drug addiction.

The term “cardiovascular diseases and disorders” preferably refers to hypertension, cardiac arrhythmias, atrial fibrillation, congenital heart failure, heart block.

The term “cancer” refers to skin cancer including melanoma including metastatic melanoma; lung cancer including non-small cell lung cancer; bladder cancer including urinary bladder cancer, urothelial cell carcinoma; renal carcinomas including renal cell carcinoma, metastatic renal cell carcinoma, metastatic renal clear cell carcinoma; gastro-intestinal cancers including colorectal cancer, metastatic colorectal cancer, familial adenomatous polyposis (FAP), esophageal cancer, gastric cancer, gallbladder cancer, cholangiocarcinoma, hepatocellular carcinoma, and pancreatic cancer such as pancreatic adenocarcinoma or pancreatic ductal carcinoma; endometrial cancer; ovarian cancer; cervical cancer; neuroblastoma; prostate cancer including castrate-resistant prostate cancer; brain tumors including brain metastases, malignant gliomas, glioblastoma multiforme, medulloblastoma, meningiomas; breast cancer including triple negative breast carcinoma; oral tumors; nasopharyngeal tumors; thoracic cancer; head and neck cancer; leukemias including acute myeloid leukemia, adult T-cell leukemia; carcinomas; adenocarcinomas; thyroid carcinoma including papillary thyroid carcinoma; choriocarcinoma; Ewing's sarcoma; osteosarcoma; rhabdomyosarcoma; Kaposi's sarcoma;
lymphoma including Burkitt's lymphoma, Hodgkin's lymphoma, MALT lymphoma; multiple myelomas; or virally induced tumors.

The pharmaceutical compositions as defined in embodiments 1) to 19) are also useful in a method of reducing the concentration of calcium in a neuronal cell, and wherein said reduction in calcium is achieved by blocking the calcium T-channel present in such neuronal cell; said method comprising the administration of the pharmaceutical compositions as defined in embodiments 1) to 19).

The pharmaceutical compositions as defined in embodiments 1) to 19) are also useful in a method of decreasing burst firing discharges in a neuronal cell and wherein said decrease of burst firing is achieved by blocking the calcium T-channel; said method comprising the pharmaceutical compositions as defined in embodiments 1) to 19).

For avoidance of any doubt, if compositions are described as useful for the prevention or treatment of certain diseases or disorders, such compositions are likewise suitable for use in the preparation of a medicament for the prevention or treatment of said diseases; and suitable for use in a method of preventing or treating said diseases comprising administering to a subject (notably a mammal, especially a human) in need thereof a pharmaceutically active amount of said compound/composition.

The term “prevent(s)” or “prevention(s)” or “preventing” used with reference to a disease means either that said disease does not occur in the patient or animal, or that, although the animal or patient is affected by the disease, part or all the symptoms of the disease are either reduced or absent. The terms “prevention” may also be understood to mean “prophylaxis”.

The term “treat(s)” or “treatment(s)” or “treating” used with reference to a disease means either that said disease is cured in the patient or animal, or that, although the animal or patient remains affected by the disease, part or all the symptoms of the disease are either reduced or eliminated.

The following examples are provided to further illustrate the invention. These examples are illustrative only and should not be construed as limiting the invention in any way.

### Examples

Raw materials can be purchased from commercial suppliers and can be used as received without further purification: Avicel™ PH-101 can be purchased from FMC; Starch 1500™ can be purchased from Colorcon; Kollidon 30™ can be purchased from BASF; Vitamin E TPGS can be purchased from Antares Health Products; Kollidon 30™ can be purchased from BASF Aerosil™ 200 can be purchased from Evonik Industries AG; Parteck™ LUB MST can be purchased from Merck KGaA; AquaPolish™ P white MS can be purchased from Biogrund GmbH.

All temperatures are stated in ° C.

Abbreviations
Ex. Example
Ref. Reference
g gram(s)
HPLC High Performance Liquid Chromatography
HSG high-shear granulator/granulation
IPC In-Process-Controls
kN kilonewton
L liter(s)
LOD loss on drying
mg milligram(s)
μL microliter(s)
min minute(s)
mL milliliter(s)
mm millimeter(s)
mM millimolar
rpm revolutions per minute
RT room temperature
SLS sodium lauryl sulfate
UPLC Ultra-Performance Liquid Chromatography

Solid pharmaceutical formulations for oral use compressed in mini-tablets

The pharmaceutical compositions of Examples 1 to 3 and Reference Examples 4 to 6 (ingredients indicated in Table 1 below) were compressed in mini-tablets (average total tablet weight of 7 mg).

TABLE 1

| Ingredients (in ww % | | | | Ref. | Ref. | Ref. | |
|---|---|---|---|---|---|---|---|
| of total composition) | Ex. 1 | Ex. 2 | Ex. 3 | Ex. 4 | Ex. 5 | Ex. 6 | Function |
| Inner phase | | | | | | | |
| COMPOUND (amounts given | 28.57 | 25.97 | 28.57 | 28.57 | 28.57 | 28.57 | Active |
| for the free base) | | | | | | | ingredient |
| Microcrystalline cellulose | 33.43 | 29.22 | 25.93 | 30.93 | 35.64 | 35.93 | Filler |
| (Avicel ™ PH-101) | | | | | | | |
| Partially pregelatinized maize | 15.00 | 12.52 | 15.00 | 15.00 | 15.00 | 15.00 | Disintegrant |
| starch (Starch 1500 ™) | | | | | | | |
| Povidone (Kollidon 30 ™) | 5.00 | 4.55 | 5.00 | 5.00 | 5.00 | 5.00 | Binder |
| Vitamin E TPGS | 2.50 | 4.55 | 10.00 | | | | Surfactant |
| Poloxamer 188 | | | | 5.00 | | | Surfactant |
| Sodium lauryl sulfate (SLS) | | | | | 0.29 | | Surfactant |
| Outer phase | | | | | | | |
| Partially pregelatinized maize | 5.00 | 4.55 | 5.00 | 5.00 | 5.00 | 5.00 | Disintegrant |
| starch (Starch 1500 ™) | | | | | | | |
| Microcrystalline cellulose | 5.00 | 4.55 | 5.00 | 5.00 | 5.00 | 5.00 | Filler |
| (Avicel ™ PH-102) | | | | | | | |
| Povidone (Kollidon 30 ™) | 5.00 | 4.55 | 5.00 | 5.00 | 5.00 | 5.00 | Disintegrant |
| Hydrophilic fumed silica | 0.25 | 0.23 | 0.25 | 0.25 | 0.25 | 0.25 | Glidant |
| (Aerosil ™ 200) | | | | | | | |
| Magnesium stearate | 0.25 | 0.23 | 0.25 | 0.25 | 0.25 | 0.25 | Lubricant |
| (Parteck ™ LUB MST) | | | | | | | |
| Coating | | | | | | | |
| AquaPolish ™ P white MS | | 9.09 | | | | 9.09 | Coating |
| | | | | | | | agent |
| Total (ww %) | 100.0 | 100.0 | 100.0 | 100.0 | 100.0 | 100.0 | |

All mini-tablets were manufactured following the steps depicted in Scheme I below utilizing known in the art manufacturing equipment. The mini-tablets of Examples 1 and 3 as well as Reference Examples 4 and 5 differ in their manufacturing process in that the coating step was omitted.

ACT-709478, also known as NBI-827104, represents N-[1-(5-cyano-pyridin-2-ylmethyl)-1H-pyrazol-3-yl]-2-[4-(1-trifluoromethyl-cyclopropyl)-phenyl]-acetamide.

Protocol

The internal phase raw materials (ACT-709478, microcrystalline cellulose (Avicel PH-101™), pregelatinized starch (Starch 1500™), and povidone (Kollidon 30™) are hand sieved on 800 mm sieve and introduced in a High Shear Granulator (6 liters bowl) and blended for about 3 minutes using an impeller speed of 200 rpm. Suitable amount of water is preheated at approximatively 50° C. Vit E TPGS (including a 20% overage) is melted and stirred at 50° C., whereupon it is introduced together with the preheated water in the spraying container to form the spraying solution.

This solution is sprayed on the prepared blend in the High Shear Granulator under constant mixing. The spray rate is adjusted to reach a spraying duration of about 2 minutes. To ensure that the right amount of Vitamin E TPGS was sprayed on the dry blend, the spraying container is weighted before and after the spraying step.

The kneading step is conducted in High Shear Granulator for approximatively 3 min with an impeller speed of 200 rpm and a chopper speed of 1500 rpm.

As soon as the wet granules preparation is completed, the wet granules are hand sieved through a 2.5 mm sieve and manually transferred into the Fluid Bed Drier and dried with an inlet air temperature of 65° C. until an LOD value between 2.0 and 3.0%.

The external phase raw materials (Microcristalline cellulose (PH-102™), Pregelatinized starch (Starch 1500™), Povidone (Kollidon™) and hydrophilic fumed silica (Aerosil 200™) are hand sieved on 800 mm sieve and are introduced together with the dried sieved granules in a container for blending. The blending step is performed for 10 min at 32 rpm.

The lubricant (magnesium stearate) is then added in the container to the blend prepared and the lubrication step is performed (3 min at 32 rpm).

The lubricated blend is then transferred in the hopper of a rotary tableting machine equipped with punches of 2 mm diameter and mini-tablets are compressed at a 4 kN compression force, targeting a mean weight of 7 mg per mini-tablet corresponding to 2 mg of drug substance per mini-tablet. During this step, In-Process-Controls (IPC) are performed on a regular basis to ensure that the mini-tablets are compliant with the predefined specifications.

The coating suspension is prepared by introducing 10% w/w Aquapolish white (ready to use coating preparation) in 90% w/w of purified water at RT. Once the suspension is prepared, it is hand sieved on 800 mm sieve. The suspension is kept under gentle stirring during the whole coating process.

The uncoated mini-tablets are introduced either in a fluid bed coater equipped with a Wurster device or in a rotating pan coater. The coating suspension is sprayed on the mini-tablets that are simultaneously heated by hot air. Once the targeted weight gain per coated mini-tablet is reached, the spraying step is stopped, the coated mini-tablets are cooled and removed from the coater.

High-density-polyethylene (HDPE) bottles are filled with a predefined number of mini-tablets.

Dissolution Tests

Dissolution Apparatus and Working Conditions

SOTAX USP II or equivalent, 6×500 ml dissolution vessels and 6 paddles; Temperature: 37° C.; 500 mL FaSSI F (pH=6.5) in each vessel 1, 2, 3, 4, 5 and 6; No sinker; Stirring: Paddles, 75 rpm; Sampling time points: 5, 10, 15, 30, 45, 60, 90, 120 min, and infinity (i.e. additional stirring for 15 min at 120 rpm); Automatic sampling with glass fiber filter 1 μm; Sample volume: 1 mL; No replacement of the sample volume with dissolution media;

UPLC Equipment and Working Conditions

Ultra-Performance Liquid Chromatography H-Class from Waters Corporation with a stationary phase (column): UPLC ACQUITY BEH SHIELD RP18 1.7 μm 75 mm and PDA Detector Acquity UPLC; Column temperature 40° C.; Mobile phase: 0.05% formic acid in acetonitrile (B) and 0.05% formic acid in water (A); Flow rate: 0.5 mL/min with gradient; Injection volume: 3 μL; Detection: UV-VIS1: 250 nm (UV-VIS2: 222 nm, UV-VIS3: 310 nm); Autosampler temperature: 25° C.; Gradient:

| Time (min) | % solvent A | % solvent B |
|---|---|---|
| 0.00 | 95.0 | 5.0 |
| 4.50 | 5.0 | 95.0 |
| 5.00 | 0.0 | 100.0 |
| 5.50 | 0.0 | 100.0 |
| 6.00 | 95.0 | 5.0 |
| 9.00 | 95.0 | 5.0 |

Preparation of dissolution medium-Fasted State Simulated Intestinal Fluid (FaSSIF), pH 6.5

FaSSIF (pH=6.5) can also be prepared by known in the art methods. For example, 2.1 g sodium hydroxide, 19.77 g sodium dihydrogen phosphate and 30.93 g NaCl were weighed into a suitable volumetric flask and about 90% of the final volume of purified water was added. The mixture was stirred for about 5 min. The flask was filled up to 5 L and then stirred for an additional 5 min to give Solution 1. The same procedure was repeated with 0.42 g sodium hydroxide, 3.95 g sodium dihydrogen phosphate and 6.19 g NaCl, wherein the flask was filled up to 1 L to give Solution 2. Solution 1 and 2 were mixed to produce 6 L of phosphate buffer. 90% of the phosphate buffer was filled into a suitable flask. 4.64 g of the product FaSSIF/FeSSIF/FaSSGF (a mixture of sodium taurocholate and soybean lecithin comprising sodium taurocholate >66.5%, phospholipids >23.5%, total surfactant content >90%; supplier: www.biorelevant.com) was added and the solution stirred for one hour. The flask was filled up to 2 L with phosphate buffer to give Solution 3. The same procedure was repeated with 2.32 g of the same FaSSIF/FeSSIF/FaSSGF powder and diluted up to 1 L to give Solution 4. Solutions 3 and 4 were mixed to produce 3 L of FaSSIF (pH=6.5) comprising 3 mM taurocholate, 0.75 mM phospholipids, 148 mM sodium, 106 mM chloride and 29 mM phosphate.

Protocol

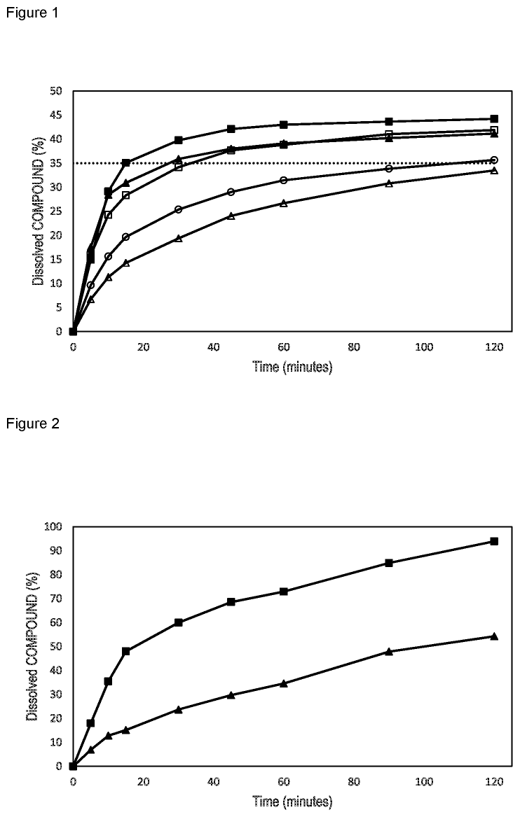

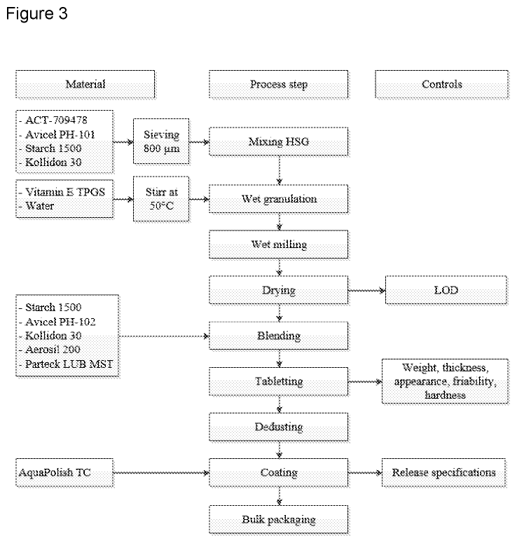

Dissolution experiments were performed in accordance with United State Pharmacopeia (USP) chapters NF711 and NF1094. Either one mini-tablet (1×2 mg of COMPOUND; Example 2 and Reference Example 6) or five mini-tablets (5×2 mg of COMPOUND; Examples 1 to 3 and Reference Examples 4 and 5) per vessel were tested according to the dissolution method described hereinabove. An aliquot of 500 μL of each dissolution sample at the corresponding sampling time point was diluted with 500 μL methanol, mixed by vortex and analyzed using the UPLC method described hereinabove. The results are shown in FIGS. 1 and 2.

The comparative test results show that the selection of the particular surfactant in the pharmaceutical compositions according to the present invention leads to advantageous in vitro dissolution properties of said compositions.

Clinic Trial for ACT-709478

Title of study: Phase 2 Multicenter, Randomized, Double-Blind, Placebo-Controlled, Parallel-Group Study to Assess the Efficacy, Safety, Tolerability, and Pharmacokinetics of NBI-827104 in Pediatric Subjects with Epileptic Encephalopathy with Continuous Spike-and-Wave During Sleep

Methodology: This is a Phase 2, multicenter, double-blind, placebo-controlled, parallel-group study designed to assess the efficacy, safety, tolerability, and PK of NBI-827104 in pediatric subjects with EECSWS. Approximately 24 male and female subjects, 4 to 12 years of age (inclusive), will be enrolled for study participation. Treatment will be administered for up to 13 weeks including titration and taper periods.

After providing parental or legal guardian informed consent with signed and witnessed pediatric assent from developmentally capable pediatric subjects, subjects will be screened to determine eligibility (Days −28 to −1) before the start of study treatment dosing on Day 1. Caregiver(s) will be given a seizure diary at screening, Day 1, and Weeks 2, 3, 4, 6, 8, 10, 12, and 13, and caregiver(s) will return the seizure diary at the next scheduled visit. On Day 1, eligible subjects who have a diagnosis confirmed by an external Diagnosis Confirmation Panel (DCP) will return to the study center for collection of baseline safety and efficacy assessments. Subjects who are confirmed as eligible will then be randomized to NBI-827104 or placebo. The first 6 subjects (randomized 2:1; ie, 4 subjects randomized to NBI-827104 and 2 subjects randomized to placebo) will be enrolled into a sentinel cohort for the analysis of safety, tolerability, and PK data through the end of Week 6, followed by the remaining 18 subjects in the main cohort (randomized 2:1 [NBI-827104:placebo]). Study treatment will last for up to 13 weeks, and will be subdivided as follows:

Titration period of 3 weeks (initial dose on Day 1 and weekly dose increases on Day 8 and Day 15) Maintenance period from the beginning of Week 4 to end of Week 12 Taper period of 1 week (Week 13) with dose reduced by 1 dose level from the maintenance period dose (subjects who received dose level 1 during maintenance will discontinue treatment at the end of Week 12 and not have a taper period)

The starting dose of study treatment for the sentinel cohort (dose level 1) will be based on body weight categories (based on subjects' body weight on Day 1), as detailed in the table below. This study intends to target exposures equivalent to those observed with a 60 mg once-daily dose at steady state in adults (area under the concentration versus time curve during 1 dosing interval [AUCT] of 13.7 μgxh/mL [95% confidence interval (CD: 11.2, 16.7 μgxh/m14. Based on the exposure data obtained from the sentinel cohort, the doses may be adjusted if required to achieve the targeted exposure in the main cohort.

| Body Weight | Dose Level 1 | Dose Level 2 | Dose Level 3 |
|---|---|---|---|
| (kg) | (mg) | (mg) | (mg) |
| ≥10 and ≤15 | 2 | 6 | 12 |
| >15 and ≤35 | 4 | 12 | 24 |
| >35 and ≤55 | 6 | 18 | 36 |
| >55 | 10 | 30 | 60 |

Doses will be administered at home and dose increases will occur at weekly clinic visits during the titration period. The dose will be increased during clinic visits at the beginning of Week 2 (dose level 2) and the beginning of Week 3 (dose level 3); dose level 3 will be maintained up to the end of Week 12. If a subject cannot tolerate a dose after dose escalation, he/she can receive the next lower tolerated dose level and remain at that dose for the remainder of the study. Subjects who cannot tolerate the lowest allowable dose (ie, dose level 1) should remain in the study, but study treatment dosing will be discontinued.

At the Week 12 visit, subjects will receive study treatment to administer at home during the taper period. To avoid potential rebound effects after abrupt treatment discontinuation, the dose of study treatment will be 1 dose level lower than the last dose level the subject received during the maintenance period. Subjects who are receiving dose level 1 at Week 12 will discontinue study treatment and enter the 4-week posttreatment safety period, which will end at Week 16. For all other subjects, the end of Week 13 is the end of the treatment period, and subjects will then enter the posttreatment safety period, which will end at Week 17. During the posttreatment safety period, subjects/caregivers will be instructed to:
Contact the investigator (or designee) if symptoms worsen and require medical attention
Report any adverse events (AEs) or serious adverse events (SAEs)

The end of the posttreatment safety period constitutes the end of the study.

Overnight video-EEGs will be performed at screening, end of Week 6, end of Week 12, or early termination for subjects who discontinue before the end of Week 6. For the Week 6 visit, subjects must arrive at the study site prior to the morning dose of study treatment and remain at the study site for the entire 24-hour PK-collection period, including the overnight video-EEG.

Safety and tolerability assessments including AE monitoring, clinical laboratory tests (including hematology, serum chemistry, and urinalysis), vital signs measurements, physical and neurological examinations, 12-lead electrocardiograms (ECGs), and ophthalmic examinations will be conducted throughout the study.

Ongoing review of safety and tolerability data and the unblinded analysis of sentinel cohort data (including safety and tolerability) will be conducted by the Independent Data Monitoring Committee (IDMC); sentinel cohort data (including PK data) will be evaluated before proceeding to the main cohort. The IDMC has the overall responsibility of safeguarding the interests of the subjects by monitoring data obtained in the study and making appropriate recommendations based on the reported data, thus ensuring that the study is being conducted with high scientific and ethical standards. Study site personnel and the Sponsor (except for supply chain personnel not involved in decisions regarding subject treatment) will remain blinded.

Available PK and safety data for the sentinel cohort will be reviewed by the IDMC to determine if a dose adjustment should be instituted prior to enrolling the main cohort. Dose reductions or alterations in the escalation paradigm may be recommended by the IDMC to the Sponsor after the sentinel cohort if the drug is not sufficiently tolerated. In addition, dose reductions or escalations may be instituted if the median exposure values for the sentinel cohort lie outside the 5th to 95th percentiles of the simulated reference range across the PK sampling intervals.

All study visits after Day 1 will have a visit window of ±3 days.

Various modifications of the embodiments, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A solid pharmaceutical composition comprising, as active ingredient, N-[1-(5-cyano-pyridin-2-ylmethyl)-1H-pyrazol-3-yl]-2-[4-(1-trifluoromethyl-cyclopropyl)-phenyl]acetamide or a pharmaceutically acceptable salt thereof, wherein said composition further comprises, as surfactant, tocopherol polyethylene glycol succinate.

2. The composition according to claim 1, wherein the active ingredient is from about 5 to about 60 ww % of the total weight of composition.

3. The composition according to any one of claim 1 or 2, wherein the surfactant is α-tocopherol polyethylene glycol succinate.

4. The composition according to any one of claims 1 to 3, wherein the surfactant is from about 0.1 to about 20 ww % of the total weight of the composition.

5. The composition according to any one of claims 1 to 4, further comprising one or more fillers.

6. The composition according to any one of claims 1 to 5, further comprising one or more disintegrants.

7. The composition according to any one of claims 1 to 6, further comprising one or more binders.

8. The composition according to claim 1 comprising
from about 20 to about 30 ww % N-[1-(5-cyano-pyridin-2-ylmethyl)-1H-pyrazol-3-yl]-2-[4-(1-trifluoromethyl-cyclopropyl)-phenyl]acetamide or a pharmaceutically acceptable salt thereof;
from about 0.5 to about 10 ww % D-α-tocopherol polyethylene glycol 1000 succinate;
from about 20 to about 40 ww % microcrystalline cellulose;
from about 1 to about 10 ww % polyvinylpyrrolidone;
from about 5 to about 25 ww % partially pregelatinized maize starch;
from 0 to about 3 ww % hydrophilic fumed silica;
from 0 to about 3 ww % magnesium stearate; and
from 0 to about 15 ww % coating agent;
wherein the total of said pharmaceutical composition is 100 ww %.

9. The composition according to claim 1 consisting essentially of
from about 20 to about 30 ww % N-[1-(5-cyano-pyridin-2-ylmethyl)-1H-pyrazol-3-yl]-2-[4-(1-trifluoromethyl-cyclopropyl)-phenyl]acetamide or a pharmaceutically acceptable salt thereof;
from about 2 to about 6 ww % D-α-tocopherol polyethylene glycol 1000 succinate;
from about 64 to about 78 ww % one or more further excipients;
wherein the total of said pharmaceutical composition is 100 ww %.

10. The composition according to any one of claims 1 to 9, wherein said composition is in the form of a tablet, mini-tablet, micro-tablet, capsule-shaped tablet, caplet, pediatric tablet, cachet, capsule, pellet, micro-pellet, bead, pill, sphere, micro-sphere, granule, oral granule, dragee, rod or disc.

11. A pharmaceutical composition according to any one of claims 1 to 10 for use as a medicament.

12. A pharmaceutical composition according to any one of claims 1 to 10 for use in the prevention/prophylaxis or treatment of diseases and disorders related to T-type calcium channels.

13. A pharmaceutical composition according to any one of claims 1 to 10 for use in the prevention/prophylaxis or treatment of diseases and disorders selected from epilepsy; epileptic encephalopathy with continuous spike-and-wave during sleep; absence epilepsy; childhood absence; idiopathic generalized epilepsies; temporal lobe epilepsy; sleep disorders; sleep disturbances; pain; inflammatory pain; neuropathic pain; peripheral pain; chronic pain; neurological diseases and disorders; essential tremor; Parkinson's disease; schizophrenia; depression; anxiety; psychosis; neurodegenerative disorders; autism; drug addiction; cardiovascular diseases and disorders; hypertension; cardiac arrhythmias; atrial fibrillation; congenital heart failure; heart block; cancer; diabetes and diabetic neuropathy; infertility; and sexual dysfunction.

14. A pharmaceutical composition according to any one of claims 1 to 10 for use in the prevention/prophylaxis or treatment of epilepsy and/or essential tremor.

15. A pharmaceutical composition according to any one of claims 1 to 10 for use in the prevention/prophylaxis or treatment of epileptic encephalopathy with continuous spike-and-wave during sleep.
